(19) United States
(12) Patent Application Publication
(10) Pub. No.: US 2025/0160679 A1
(43) Pub. Date: May 22, 2025

(54) SMART FOOTWEAR DETECTION AND INSOLE PRESCRIPTION METHOD AND SYSTEM

(71) Applicant: DR. FOOT TECHNOLOGY CO., LTD., Yilan County (TW)

(72) Inventor: WEI-CHENG LIN, Yilan County (TW)

(73) Assignee: DR. FOOT TECHNOLOGY CO., LTD., Yilan County (TW)

(21) Appl. No.: 18/791,457

(22) Filed: Aug. 1, 2024

(30) Foreign Application Priority Data
Nov. 22, 2023 (TW) 112145203

(51) Int. Cl. A61B 5/103; A61B 5/11; G06T 7/00; G06T 7/60; G06T 7/73; G06V 10/40; G16H 20/30

(52) U.S. Cl. CPC A61B 5/1036; A61B 5/1116; A61B 5/1128; G06T 7/0012; G06T 7/60; G06T 7/73; G06V 10/40; G16H 20/30; G06T 2207/30196

(57) ABSTRACT

The present disclosure provides a smart footwear detection and insole prescription method adapted to be performed on an electronic device. The smart footwear detection and insole prescription method includes receiving foot image information, acquiring a foot posture feature based on the foot image information, and generating an insole prescription according to the foot posture feature. The present disclosure is suitable for providing a smart footwear detection and insole prescription method that can be performed easily and automatically at all times and in all places.

### CROSS-REFERENCE TO RELATED APPLICATION

This non-provisional application claims priority under 35 U.S.C. § 119(a) on Patent Application No(s). 112145203 filed in Taiwan, R.O.C. on Nov. 22, 2023, the entire contents of which are hereby incorporated by reference.

### BACKGROUND OF THE INVENTION

### 1. Field of the Invention

The present disclosure provides an insole prescription method and system, and in particular to an automatic smart footwear detection and insole prescription method and system.

### 2. Description of the Related Art

Common commercially available shoes are provided in various different sizes but only one fixed version for users to choose from, and are unable to meet current user requirements due to the lack of adaptation to specific differences of feet of individual users. Most of the users who have no access to properly fitting shoes can even suffer from symptoms of sore feet.

Moreover, common commercially available insoles are mostly made of a cushioning material in a simple form of one layer, and are used for buffering or height padding between feet and shoe bodies without providing other special functions.

In sports of various sport categories, in particular for athletes of professional competitive sports, insoles are frequently used to enhance sports performance, for example, reducing deviations, increasing ball speed and distance, reducing muscle stress, improving foot posture and reducing foot pain. In addition to enhancing sports performance, such insoles can also provide ankles and knees of the general public doing sports with effects of protection and injury prevention. Thus, insoles are considered necessary equipment of general users as well as common reserve equipment for users with health promotion needs.

### BRIEF SUMMARY OF THE INVENTION

Due to foot features of individual users, such as different lengths, arches, widths and arch positions, if customization cannot be provided for these foot features, parameters and requirements of individual users, functions of insoles cannot be fully practiced. Moreover, for insoles that reliably reduce pressure and offer relief and support, in addition to purely providing appropriate thickness and firmness in regions subject to large forces and high pressures, various special structures are also needed to provide functions of pressure reduction, buffering, support and stabilization.

In current insole customization, customized measurement and detection are frequently performed for users by professionals using special measurement apparatuses (for example, foot scanners) in professional medical facility areas and insole supplier stores, so as to provide insoles having suitable functions or having suitable measures for intended corrections. During an actual treatment process, adjustments need to be made constantly to insoles in response to treatment and improvement conditions. In conventional approaches, users are required to frequently head to medical facilities and/or insole supplier stores in order to learn and acquire conditions of their feet (for example, whether an ankle tilt has been improved) and gait habits during footwear wearing (for example, whether a force is constantly applied out of habit to a predetermined region), and accordingly acquire insole prescriptions corresponding to such adjustments.

Therefore, for individual users with an extensive range of requirements, there is a need for a solution to provide customized insoles which are properly fitting and functional and/or provide rehabilitation and health care for foot diseases and/or insole prescriptions thereof, and customized insoles which improve comfort, reduce burdens on muscles and joints and enhance sports performance, thereby reducing burdens of sports or meeting injury prevention for general users, and at the same time to enable users to conveniently and easily carry out a full footwear detection and insole prescription procedure in a comfortable household area while overcoming venue restrictions and reducing human intervention, thereby improving convenience, accessibility, reliability and efficiency in providing the insole prescription.

In view of the above, the present disclosure provides a smart footwear detection and insole prescription method and system for automatically, quickly, stably and accurately detecting and analyzing feet, footwear and subjective requirements of a user to accordingly provide properly adapted insole prescriptions, and applicable in various venues such as personal household areas. According to foot image information captured by a user or other individuals using a common electronic device, the user is able to at all times and in all places analyze and acquire foot posture features, and an insole prescription adapted to a foot of the user can be automatically generated. With the insole prescription, a customized insole meeting user requirements of pressure reduction, buffering, force dispersion, support, fixation and stability can be subsequently manufactured.

A smart footwear detection and insole prescription method adapted to be performed on an electronic device is provided according to an embodiment of the present disclosure. The smart footwear detection and insole prescription method includes receiving foot image information, acquiring a foot posture feature based on the foot image information, and generating an insole prescription according to the foot posture feature.

In the smart footwear detection and insole prescription method, the foot image information includes heel image information, the heel image information includes a heel dynamic image and a heel still image, the foot posture feature includes a heel feature parameter, and the heel feature parameter is acquired based on the heel image information.

In the smart footwear detection and insole prescription method, the foot posture feature includes an arch height parameter and an arch length parameter, the arch height parameter and the arch length parameter are acquired by an image capturing module, and a grid pattern is used as an image capturing background when the arch height parameter and the arch length parameter are acquired by the image capturing module.

In the smart footwear detection and insole prescription method, the foot posture feature includes a hallux valgus angle parameter.

In the smart footwear detection and insole prescription method, the hallux valgus angle parameter is acquired by an image capturing module, and a uniform background is used as an image capturing background when the hallux valgus angle parameter is acquired by the image capturing module.

In the smart footwear detection and insole prescription method, the foot posture feature includes at least one of a group consisting of a foot length parameter, a foot width parameter, a foot sole circumference parameter and a body posture feature.

In the smart footwear detection and insole prescription method, the operation of acquiring a foot posture feature based on the foot image information is performed by an artificial intelligence model.

The smart footwear detection and insole prescription method further includes receiving footwear detection image information, acquiring a footwear usage feature based on the footwear image information, and adjusting the insole prescription according to the footwear usage feature.

In the smart footwear detection and insole prescription method, the footwear detection image information includes a footwear heel image and a footwear bottom image.

The smart footwear detection and insole prescription method further includes receiving a user requirement input, acquiring a usage requirement feature according to the usage requirement input, and adjusting the insole prescription according to the usage requirement input.

In the smart footwear detection and insole prescription method, the insole prescription can be acquired according to the foot posture feature, a footwear usage feature and a usage requirement feature.

In the smart footwear detection and insole prescription method, the insole prescription includes a support adaptation parameter including an adaptively adjustable insole arch height parameter. The insole arch height parameter includes at least one selected from a group consisting of: a transverse arch pad parameter, which can be acquired by integration and adaption according to a foot length parameter, a foot width parameter, a foot sole circumference parameter, a body posture feature and a sports item requirement; an inner arch parameter, which can be acquired by integration and adaption according to an arch height parameter, an arch length parameter and a medical disease name requirement; an outer arch parameter, which can be acquired by adaption according to the sports item requirement; an arch pad parameter, which can be acquired by integration and adaptation according to the arch height parameter, the arch length parameter, the sports item requirement and the medical disease name requirement; and a mid-foot bridge width parameter, which can be acquired by adaptation according to a footwear type.

In the smart footwear detection and insole prescription method, the insole prescription includes an angle adaptation angle, including at least one of a group consisting of a forefoot locally heightening parameter and a rearfoot locally heightening parameter, the forefoot locally heightening parameter can be acquired by adaptation according to a hallux valgus angle parameter, and the rearfoot locally heightening parameter can be acquired by adaptation according to a heel feature parameter.

In the smart footwear detection and insole prescription method, the insole prescription includes a pressure reduction adaptation angle, including at least one of a group consisting of a forefoot reinforcement parameter and a rearfoot reinforcement parameter, the forefoot reinforcement parameter can be acquired by integration and adaptation according to a hallux valgus angle parameter and a user requirement feature, and the rearfoot reinforcement parameter can be acquired by integration and adaptation according to a heel feature parameter, a sports item requirement, a medical disease name requirement and the usage requirement feature.

In the smart footwear detection and insole prescription method, the insole prescription includes a thickness adaptation parameter, which can be acquired by adaptation according to a usage requirement feature including a sports item requirement and a medical disease name requirement.

In the smart footwear detection and insole prescription method, the insole prescription includes a heel shape adaptation parameter, which can be acquired by adaptation according to a usage requirement feature including a sports item requirement and age information.

A smart footwear detection and insole prescription system provided according to an embodiment of the present disclosure includes: a server; and an electronic device, operable to be electrically connected to the server, the electronic device including a processor and a storage device, the storage device having multiple program instructions stored therein, the processor capable of performing the steps of the smart footwear detection and insole prescription method described above after executing the program instructions.

With the smart footwear detection and insole prescription method and system of the present disclosure, by determining foot posture features in foot image information and accordingly providing an insole prescription, an insole prescription for a properly fitting and functional insole and/or providing rehabilitation and health care for foot diseases and a further customized insole can be provided. Moreover, the insole prescription with improved comfort, reduced burdens on muscles and enhanced sports performance is provided according to different factors, such as objective foot features, objective footwear features, subjective sensations and usage requirements, thereby reducing burdens of sports or meeting requirements of injury prevention for general users.

Meanwhile, the smart footwear detection and insole prescription method and system of the present disclosure enable users to conveniently and easily carry out a full footwear detection and insole prescription procedure in a comfortable household area while overcoming venue restrictions and reducing human intervention, thereby improving convenience, accessibility, reliability and efficiency in providing the insole prescription. Accordingly, the present disclosure is capable of automatically, quickly, stably and accurately detecting and analyzing feet, footwear and subjective requirements of a user to accordingly provide properly adapted insole prescriptions, and is applicable in various venues such as personal household areas. According to foot image information captured by a user or other individuals using a common electronic device, the user is able to at all times and in all places analyze and acquire foot posture features, and an insole prescription adapted to a foot of the user can be automatically generated. With the insole prescription, a customized insole meeting user requirements of pressure reduction, buffering, force dispersion, support, fixation and stability can be subsequently manufactured.

### BRIEF DESCRIPTION OF THE DRAWINGS

FIG. 1 is a flowchart of a smart footwear detection and insole prescription method according to an embodiment of the present disclosure.

FIG. 2 is a flowchart of a smart footwear detection and insole prescription method according to another embodiment of the present disclosure.

FIG. 3 is a flowchart of a smart footwear detection and insole prescription method according to another embodiment of the present disclosure.

FIG. 4 is a flowchart of a smart footwear detection and insole prescription method according to yet another embodiment of the present disclosure.

FIG. 5 is a block diagram of a structure of a smart footwear detection and insole prescription system according to an embodiment of the present disclosure.

FIG. 6 is an illustrative schematic diagram of acquiring a foot posture feature according to an embodiment of the present disclosure.

FIG. 7 is an illustrative schematic diagram of acquiring a footwear usage feature according to an embodiment of the present disclosure.

FIG. 8 is an illustrative schematic diagram of acquiring a hallux valgus angle parameter according to an embodiment of the present disclosure.

FIG. 9 is an illustrative schematic diagram of acquiring an arch height parameter and an arch length parameter according to an embodiment of the present disclosure.

FIG. 10 is an illustrative schematic diagram of acquiring a foot length parameter, a foot width parameter and a foot sole circumference parameter according to an embodiment of the present disclosure.

FIG. 11 is a schematic diagram of gait analysis of a heel dynamic image according to an embodiment of the present disclosure.

### DETAILED DESCRIPTION OF THE INVENTION

The technical contents of the present disclosure are to be further described in detail by way of embodiments with the accompanying drawings below. It should be noted that, in the present disclosure of the literature, terms such as “first”, “second” and “third” are used to distinguish differences among elements, and are not to be construed as limiting to the elements themselves or specific orders of the elements. Moreover, in the present disclosure of the literature, a specific number is specified, the article “a/an/one” refers to one element or more.

To facilitate understanding of the object, characteristics and effects of this present disclosure, embodiments together with the attached drawings for the detailed description of the present disclosure are provided.

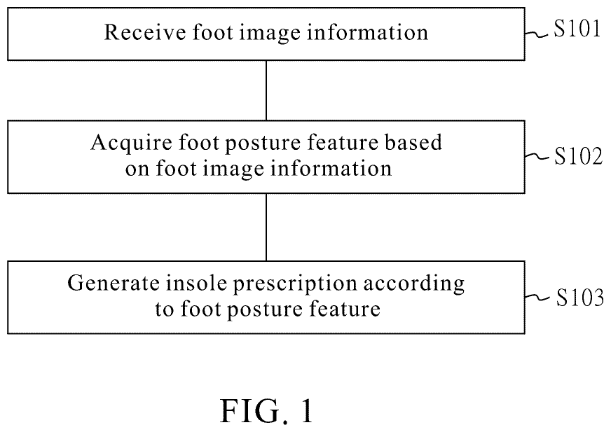

FIG. 1 shows a flowchart of a smart footwear detection and insole prescription method according to an embodiment of the present disclosure. Referring to FIG. 1, the smart footwear detection and insole prescription method provided by the present disclosure is adapted to be performed on an electronic device, and the method can be specifically completed by step S101 to step S103 shown in FIG. 1. It should be noted that the numerals of the steps are merely illustrative and are not to be construed as limitations to an operation order. The electronic device can be a smartphone, a desktop computer, a laptop computer, a personal computer, a tablet computer or a workstation. The electronic device can provide a user interface for a user to operate, and the electronic device can also indirectly perform the smart footwear detection and insole prescription method of the present disclosure via electrical and signal transmission with other electronic devices. The electronic device is suitable in any venue in which the electronic device can be used, for example, a personal household area, a store area or a medical facility area, so as to flexibly adapt to different application scenarios.

In step S101, foot image information is received. In one embodiment, when a user operates an electronic device operable to perform the smart footwear detection and insole prescription method of the present disclosure, foot image information can be acquired by an image capturing module of the electronic device, such as a lens, specifically such as a phone camera lens, a laptop computer camera lens or a webcam, or by receiving, by the electronic device, foot image information captured by other image capturing modules. In one embodiment, the foot image information includes heel image information. The heel image information includes a heel dynamic image and a heel still image; that is, one single image in a still state can be captured, or a video of multiple images or a combination of single images in a motion state can be captured.

In step S102, a foot posture feature is acquired based on the foot image information. In one embodiment, based on the foot image information, for example, pixel points of a foremost end of the sole and pixel points of a rearmost end of the sole, a distance between these pixels is a full foot length, and the foot posture in the foot image is characterized and converted to a standardized foot posture feature parameter.

In one embodiment, the heel image information can be captured in a forward direction from the rear of a foot so as to acquire a heel image from a direction of the heel, and a tilt angle of the heel is further analyzed to acquire a heel feature parameter, for example, a tilt angle and a tilt direction. The heel image information acquired can be divided into a heel still image and/or a heel dynamic image. The term “still” means that when the body is standing still, a heel posture of standing still can be obtained by capturing the heel in a forward direction from the back, and a feature parameter such as a tilt angle of the heel in a posture of standing still can be accordingly acquired. On the other hand, the term “dynamic” means that when the body is in a state of walking or running, a heel posture of the heel landing on the ground during the dynamic state of walking or running can be obtained from a video capture/recorded in a forward direction from the back. In a situation of a dynamic image in which when a non-landing foot is at a highest point, it is considered that a landing foot is in a state of receiving a maximum force. This image is used for analysis of a heel feature parameter of the heel dynamic image, and the foot posture feature under different conditions is accordingly acquired.

In one embodiment, the foot posture feature includes an arch height parameter and an arch length parameter. The arch height parameter and the arch length parameter are acquired by the image capturing module. A grid pattern is used as an image capturing background when the arch height parameter and the arch length parameter are acquired by the image capturing module. Thus, an insole prescription adapted to an arch feature of a user can be further provided, for example, a support protruding structure or a soft pad for heightening or extending the arch. Moreover, when an image related to the arch height parameter and the arch length parameter is captured, by using a grid pattern as an image capturing background, the foot posture feature of the arch height parameter and the arch length parameter can be more accurately analyzed and measured. Compared to spatial length information acquired simply by computing pixels of an image, by using a grid pattern having equidistant intervals as the image capturing background, for example, a grid pattern with continuous grids each in dimensions of 1 cm×1 cm, the grid pattern can be used as a precise reference scale. Moreover, compared to a method of using other objects as a scale, the method of using the grid pattern provided by the present disclosure is less likely affected by distances and differences, and multi-point corrections can be performed for different positions, further more accurately acquiring parameters related to the foot posture feature required.

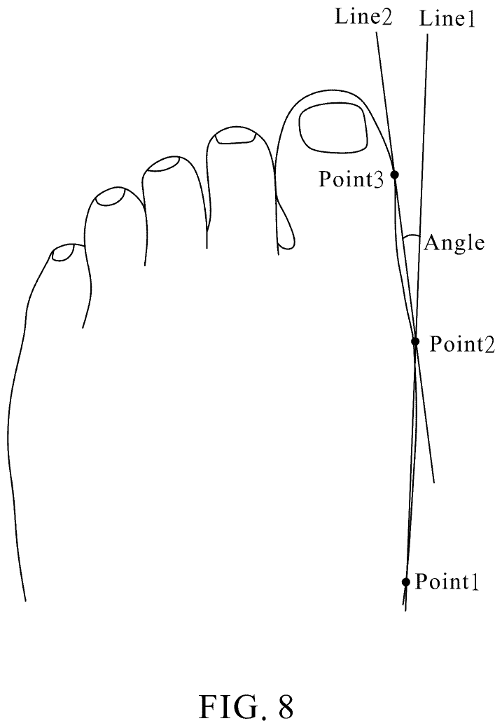

In one embodiment, the foot posture feature includes a hallux valgus angle parameter. Thus, suitable insole dimensions can be provided for a hallux valgus feature of different foot types. In one embodiment, the hallux valgus angle parameter is acquired by the image capturing module, wherein a uniform background is used as an image capturing background when the hallux valgus angle parameter is acquired by the image capturing module. Compared to the above arch height and length parameters for which a grid pattern can be used as an image capturing background, it is necessary to precisely determine an included angle between two tangents for the foot posture feature of a hallux valgus angle. Thus, a uniform background, for example, white paper or a green screen is used as an image capturing background. Thus, a profile of the sole of foot can be more accurately drawn so as to precisely determine tangents on an inner side of the sole and their tangent points, thereby more accurately acquiring the included angle between the two tangents and the hallux valgus angle thereof, with associated details to be further described with reference to FIG. 8 shortly.

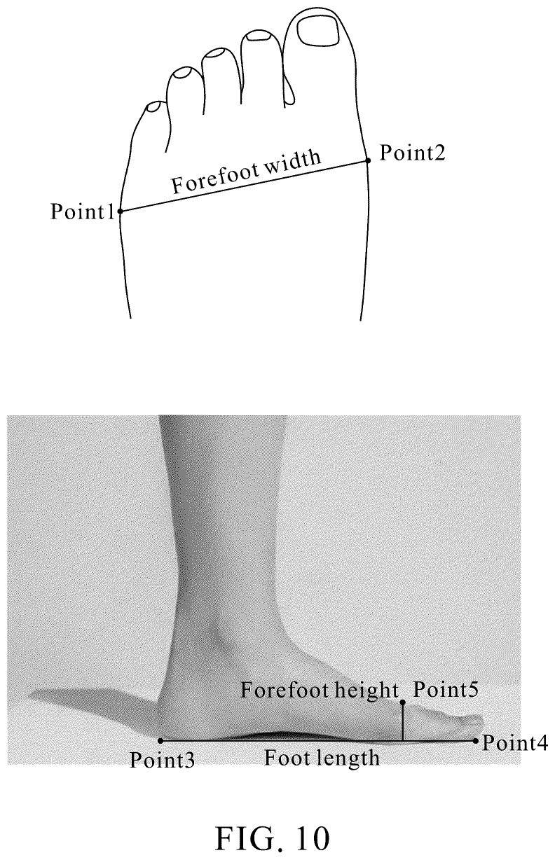

In one embodiment, the foot posture feature includes at least one of a group consisting of a foot length parameter, a foot width parameter, a foot sole circumference parameter and a body posture feature. Thus, the foot posture feature is further defined to include, for example but not limited to, the foot length parameter, the foot width parameter, the foot circumference parameter and the body posture feature, and a correspondingly adapted insole can be provided corresponding to the parameters or features. The foot length parameter refers to a full foot length of the sole, that is, a foremost end to a rearmost end of an entire foot. The foot width parameter refers to a full foot width of the sole, that is, a leftmost end to a rightmost end of an entire foot. The foot sole circumference parameter refers to a circumferential distance around a forefoot width, wherein the forefoot width refers to a connection line of protrusions of metacarpal bones, with associated details to be described with reference to FIG. 10 shortly. The body posture feature refers to a determination of whether an entire body has a tendency of side bend or hunchback from a front side and a lateral side, so as to assist in determining force receiving conditions of the sole of foot. For example, if the body posture feature indicates the presence of hunchback, it is likely that the front portion of a foot receives a greater force, and reinforcement for an insole corresponding to the forefoot needs to be correspondingly provided. In one embodiment, a process of image capturing includes partial still image capturing, full body image capturing, and dynamic gait video recording. The determining of the human posture feature for full body image capturing can be performed by skeleton recognition for further analysis, for example, positions of left and right ankles, left and right knees, left and right hip bones, left and right shoulders, left and right shoulder lines and left and right ears, can be used as determination reference points of the body posture feature.

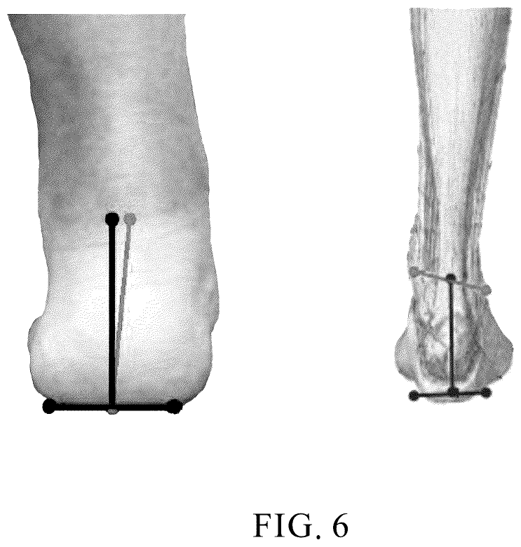

In one embodiment, the operation of acquiring a foot posture feature based on the foot image information can be performed by, for example, an artificial intelligence model such as a machine learning model or a deep learning model. An image defined with a heel tilt line is provided as training data as training reference for an artificial intelligence model. For example, as shown in FIG. 6, for a reference line of the heel tilt angle, a line at a midpoint of a line between of bone protrusions on both sides is used as a connecting line of the reference line, and an angle between this line and a line perpendicular to a horizontal bottom surface can be determined a the foot posture feature of heel tilt. With training of the artificial intelligence, uniform, standardized, automatic, fast, stable and highly repetitive interpretation for the foot posture feature can be achieved, so as to subsequently provide a stable and reliable insole prescription.

In step S103, an insole prescription is generated according to the foot posture feature. In one embodiment, after various parameters of the foot posture feature are acquired according to the above, an insole prescription for an adapted insole corresponding to correction, treatment, and use is generated. For example, when the foot posture feature indicates that the heel of a foot of a user tilts inward by 5 degrees, an adapted insole can be correspondingly provided. For example, an adapted insole having an increased thickness for an inner arch is provided to tilt the sole of foot outward by 5 degrees, so as to provide a stable support. It should be noted that, the above is merely an example, and the adaption in fact not necessarily an adaptation directly corresponding to a numeral, with associated details to be given shortly. Thus, with the insole prescription generated, a corresponding adapted insole suitable for real foot features of a user can be provided in an automatic, timely, highly repetitive, multiplexed, standardized and regular manner, so as to provide a smart customized adapted insole prescription providing effective stability, support, protection, fixation, correction, medical treatment or enhanced sports performance.

Once the insole prescription is generated, the insole prescription can be accessed by a user interface of an electronic device, provided for visual display or audio sounds, such as a display device, a touch screen, a projector, a speaker or phone audio, or accessed by other data transmission interfaces, for a user to acquire contents of the insole prescription. Moreover, related data can be further accessed or transmitted to other electronic devices or storage media. A user can also perform the above operation process of the method by a related input interactive interface, for example, by an input module of an electronic device such as a keyboard, a mouse, a touch screen, motion detection or audio recognition.

In one embodiment, data of the foot image information, the foot posture feature and the insole prescription can be stored in a hard drive or a memory of an electronic device. In one embodiment, data of the foot image information, the foot posture feature and the insole prescription can be stored in a database, a server, a cloud server, a hard drive array or other types of memories electrically connected to an electronic device performing the present method, and be transmitted to the electronic device via various electrical connections, for example, wired communications, wireless communications or the Internet. For example, in an application area for household use, the foot image information can be uploaded to a cloud computing server, analysis is performed by the cloud computing server according to the foot image information to acquire the foot posture feature, the insole prescription is generated by the cloud computing server according to the foot posture feature, and then the insole prescription is transmitted back to an electronic device of household use of the user. Thus, a user is not required to install a special program in the electronic device, and is allowed to easily perform the smart footwear detection and insole prescription method provided by the present disclosure by an image transmission means such as a simple browser interface or communication software. With the storage and access of data related to the insole prescription, flexibilities for further information discussion regarding the insole prescription with other professionals, for example, insole suppliers and medical professionals, are further reserved.

In one embodiment of the present disclosure, in the smart footwear detection and insole prescription method of the present disclosure, after the insole prescription is generated, a specific operation for outputting an insole can be further provided, including, for example but not limited to, generating an actual adapted insole product according to the insole prescription by means of 3D CNC processing, 3D printing and manual fabrication. After acquiring the insole prescription, a user, by direct ordering, can acquire services of the convenient and highly efficient insole prescription, is allowed to acquire the adapted insole prescription in a one-stop manner, understand his/her own foot features, acquire suitable the adapted insole prescription, and further directly continue production of the adapted insoles, providing effects of being convenient, fast and efficient.

Thus, after a user inputs various foot-related images and/or data of various items of foot evaluation via an electronic device, an insole prescription can be automatically generated by online computing using a smart footwear detection and insole prescription system, and then data of the insole prescription can be directly transmitted to a smart production line for start a manufacturing process of insoles. For example, regarding insole prescriptions produced by such system according to design logics, general individuals require approximately 2.2 billion prescriptions, which consist of 4 insole types, 27 sizes, various foot issues, functional requirements and insole material variations in 16 large categories and 83 small categories. With the smart footwear detection and insole prescription method and system constructed in coordination with an artificial intelligence model, manual evaluation time can be significantly reduced and manual errors can be avoided, hence reducing requirements for professional manpower and enhancing efficiency. An integration with an online artificial intelligence can also be performed. A user in a household area is allowed to perform a series of smart footwear detection and professional evaluation by an electronic device to acquire professional insole prescription and customized insoles.

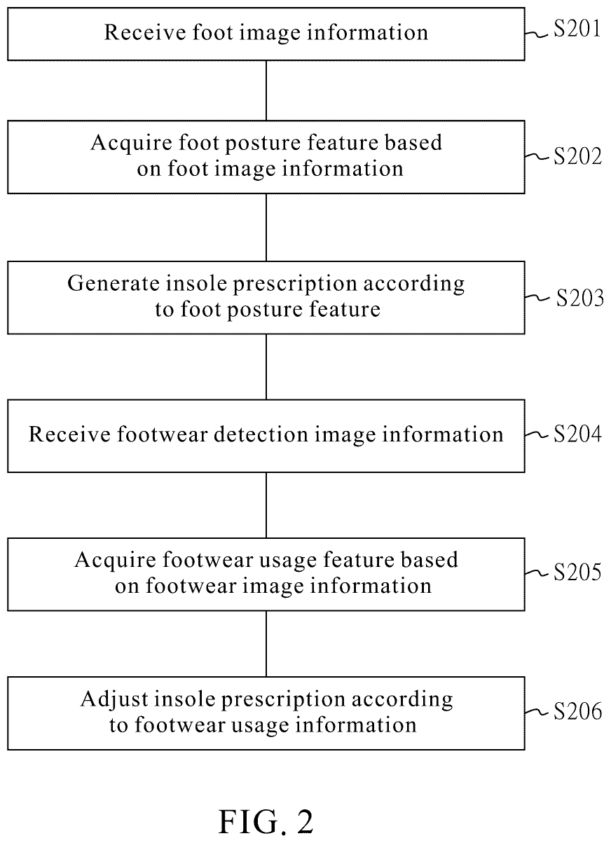

FIG. 2 shows a flowchart of a smart footwear detection and insole prescription method according to another embodiment of the present disclosure. Referring to FIG. 2, the smart footwear detection and insole prescription method provided by the present disclosure is adapted to be performed on an electronic device, and the method can be specifically completed by step S201 to step S206 shown in FIG. 2. Details of steps S201 to S203 are fundamentally respectively and sequentially the same as steps S101 to S103 in FIG. 1. It should be noted that the numerals of the steps are merely illustrative and are not to be construed as limitations to an operation order.

In step S204, footwear detection image information is received. With the image capturing module or image capturing method above, in addition to capturing the foot of a user, detection image information of a footwear used by the user can also be captured and/or received, for example, an image of a shoe usually worn by the user.

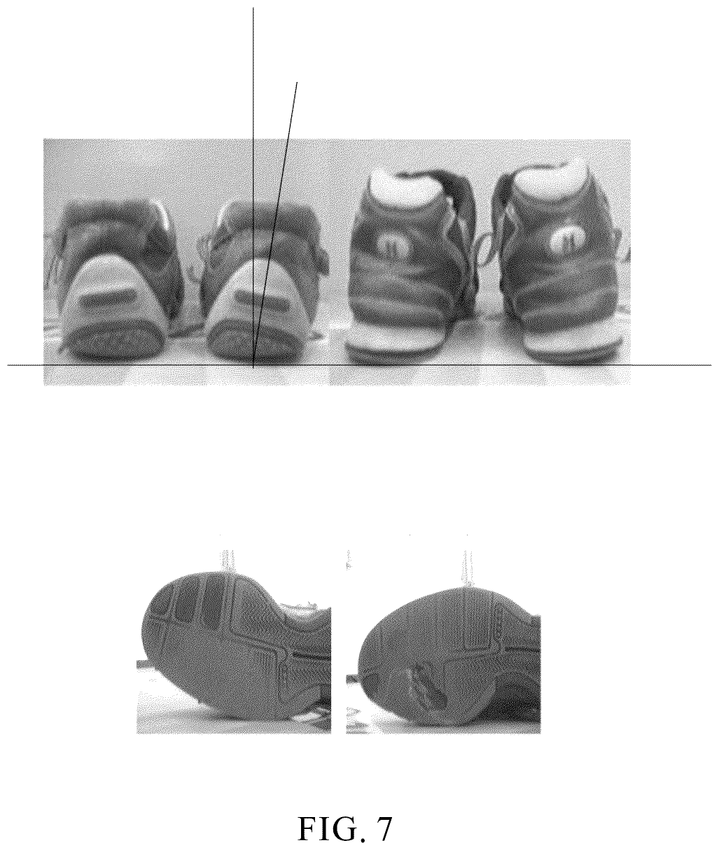

In step S205, a footwear usage feature is acquired based on the footwear image information. In one embodiment, the footwear detection image information includes a footwear heel image and a footwear bottom image. The footwear heel image can be captured by a same angle of view or method as that for the heel image so as to capture wear conditions of a heel bottom of the shoe. As shown in FIG. 7, for example, if a user has an ankle tilting outward out of habit while walking, the outer bottom of the footwear is worn out more seriously, such that the footwear heel image presents a footwear usage feature of tilting outward. The footwear bottom image is acquired by capturing the bottom of a footwear, and by wear conditions of texture such as an anti-skid pattern at the bottom of the footwear, habits of gait of the user and the footwear usage feature thereof can be learned. In one embodiment, analysis of the footwear usage feature can divide the footwear bottom image into multiple regions or quadrants, so as to qualitative or quantitative analyze levels of wear in the different regions. For example, when a user tends to wear the outer side of a footwear out of habits, the texture on the outer side of the footwear bottom is more worn out.

In step S206, the insole prescription is adjusted according to the footwear usage feature. Thus, in addition to generating the insole prescription according to the foot posture feature, an adjustable and adapted insole prescription can be further provided in combination with the footwear usage feature.

In one embodiment, the operation of acquiring a footwear usage feature based on the footwear detection image information can be performed by, for example, an artificial intelligence model such as a machine learning model or a deep learning model. An image defined with a footwear heel tilt line and an image of wear at the footwear bottom are provided as training data as training reference for an artificial intelligence model. For example, as shown in FIG. 7, for a reference line of the footwear heel tilt angle, a connecting line from a midpoint of a line at a footwear bottom to an upper edge of the footwear can be used as a reference line, and an angle between this reference line and a line perpendicular to a horizontal bottom surface can be determined as the footwear usage feature of footwear heel tilt. Moreover, for example, the image of wear at the footwear bottom is divided in regions and quadrants by an artificial intelligence model, and regions and levels of wear at the footwear bottom can be further accurately identified. With training of the artificial intelligence, uniform, standardized, automatic, fast, stable and highly repetitive interpretation for the footwear usage feature can be achieved, so as to subsequently provide a stable and reliable insole prescription.

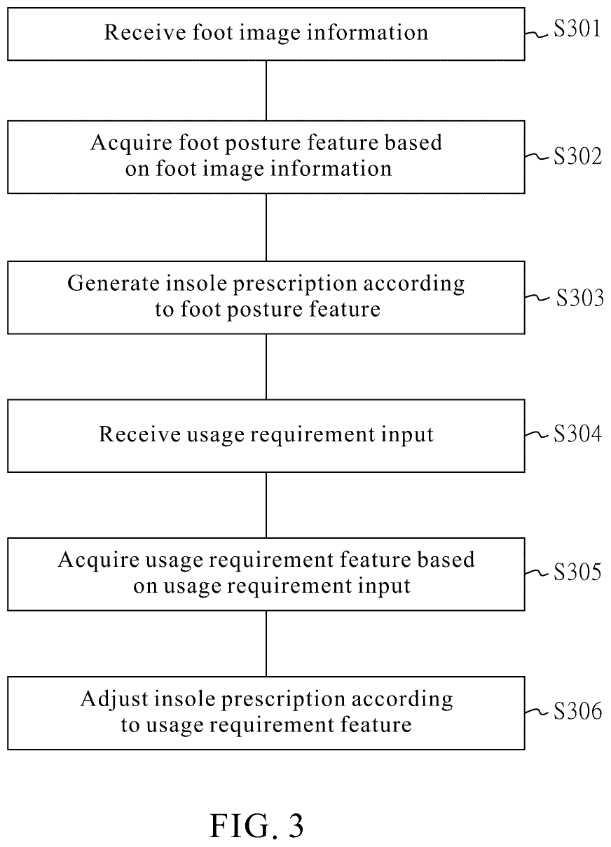

FIG. 3 shows a flowchart of a smart footwear detection and insole prescription method according to another embodiment of the present disclosure. Referring to FIG. 3, the smart footwear detection and insole prescription method provided by the present disclosure is adapted to be performed on an electronic device, and the method can be specifically completed by step S301 to step S306 shown in FIG. 3. Details of steps S301 to S303 are fundamentally respectively and sequentially the same as steps S101 to S103 in FIG. 1. It should be noted that the numerals of the steps are merely illustrative and are not to be construed as limitations to an operation order.

In step S304, a usage requirement input is received. In one embodiment, the smart footwear detection and insole prescription method of the present disclosure can further receive a usage requirement input of a user, and input information of an insole requirement of the user can be received by the user interface or input module above.

In step S305, a usage requirement feature is acquired based on the user requirement input. The usage requirement input includes, for example but not limited to, a sports item requirement, a medical disease name requirement, a footwear type, age information, and subjective sensations of a usage requirement feature, and subjective and objective requirement information of the user can be acquired in the form of a questionnaire of multiple choices or question and answer.

In step S306, the insole prescription is adjusted according to the usage requirement feature. In one embodiment, an adapted insole prescription is provided according to the sports item requirement, for example, adaptation for a narrower pattern is needed for soccer and adaptation for a wider pattern is needed for basketball. In one embodiment, an insole prescription for correction is provided according to the medical disease name requirement, for example, different correction and adapted insole prescriptions are provided for disorders such as flatfoot, plantar fasciitis, hallux valgus, high arches, genu varum (bow-shaped legs), and genu valgum (knocked knees). In one embodiment, the footwear types are available in patterns in different widths, for example, a pattern in a narrower width is provided for high-heels. In one embodiment, footwear sizes can be divided into two or more intervals according to the age information for a preliminary differentiation, for example, footwear sizes for ages between 3 and 14 are commonly in European sizes 21 EU to 32 EU, and footwear sizes for ages over 15 are commonly in European sizes 33 EU to 47 EU. In one embodiment, the subjective sensations of the usage requirement feature including whether there is pain or calluses on the forefoot, whether there is pain on the rearfoot, or which parts of the footwear are subjectively considered as being worn more seriously, are also collected and used as user subjective information for adjustment and adaptation for insole prescriptions as compared to the feature determination of other objective images.

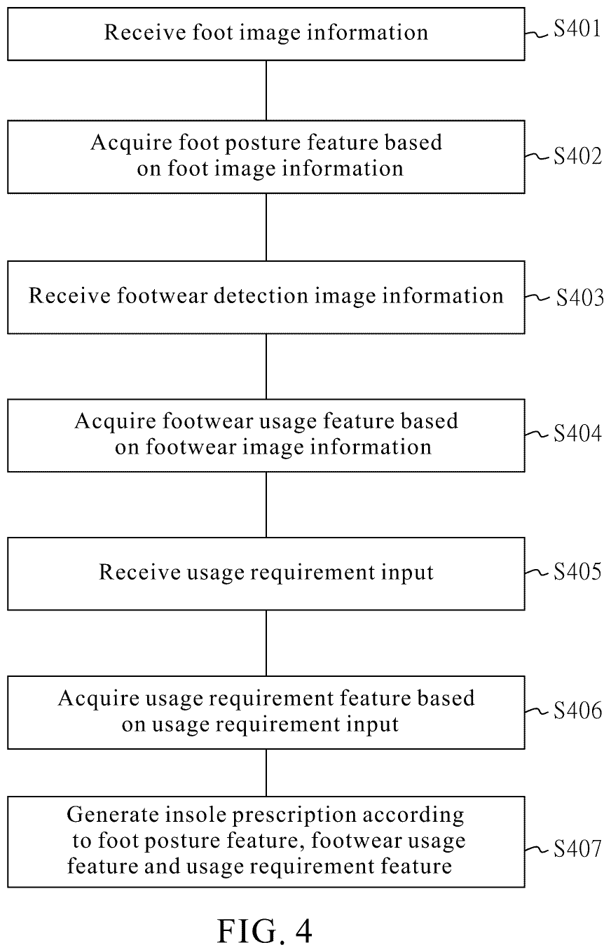

FIG. 4 shows a flowchart of a smart footwear detection and insole prescription method according to yet another embodiment of the present disclosure. Referring to FIG. 4, the smart footwear detection and insole prescription method provided by the present disclosure is adapted to be performed on an electronic device, and the method can be specifically completed by step S401 to step S407 shown in FIG. 4. The details of step S401 and step S402 are fundamentally respectively and sequentially the same as steps S101 and S102 in FIG. 1; the details of step S403 and step S404 are fundamentally respectively and sequentially the same as steps S204 and S205 in FIG. 2; the details of step S405 and step S406 are fundamentally respectively and sequentially the same as steps S304 and S305 in FIG. 3. It should be noted that the numerals of the steps are merely illustrative and are not to be construed as limitations to an operation order.

In step S407, the insole prescription is generated according to the foot posture feature, the footwear usage feature and the usage requirement; that is, all the factors above as the basis are used as factors for providing the insole prescription, so as to provide the basis that attends to both objective smart footwear detection and subjective user requirements, thereby effectively and automatically providing a user with the insole prescription meeting objective real requirements and subjective desired requirements, and achieving the smart footwear detection and insole prescription method that is truly customized.

Details of the features and factors as the basis for providing the insole prescription are further described below. For clear illustration, the various factors above are assigned with, for example, codes, for clear representation:
(A1) heel feature parameter;
(A2) hallux valgus angle parameter;
(A3) arch height parameter, arch length parameter;
(A4) foot length parameter, foot width parameter, foot sole circumference parameter;
(A5) body posture feature;
(C1) sports item requirement;
(C2) medical disease name requirement;
(C3) footwear type;
(C4) age information; and
(C5) usage requirement feature.

Out of the above, A1 to A5 can be acquired from images captured by the image capturing or from captured image received by the electronic device. C1 to C5 can be input in forms of question and answer, multiple choice or filling in by a user via a user interface or an input module of the electronic device. Predetermined options, such as sports item options, a medical disease name options, footwear type options and age options can be provided, and a user is also allowed to write down symptoms or occurring positions of discomfort to provide a link to functions of the insole prescription. For example, a user is further allowed to write down the usual duration of exercise, preferred sports types, customary footwear, customary dressing habits and preferred feet, as the basis for evaluation of the usage requirement feature.

In one embodiment, the insole prescription includes (D1) a support adaptation parameter including an adaptively adjustable insole arch height parameter. The insole arch height parameter includes at least one selected from a group consisting of: a transverse arch pad parameter, which can be acquired by integration and adaption according to (A4) the foot length parameter, (A4) the foot width parameter, (A4) the foot sole circumference parameter, (A5) the body posture feature and (C1) the sports item requirement; an inner arch parameter, which can be acquired by integration and adaption according to (A3) the arch height parameter, (A3) the arch length parameter and (C2) the medical disease name requirement; an outer arch parameter, which can be acquired by adaption according to (C1) the sports item requirement; an arch pad parameter, which can be acquired by integration and adaptation according to (A3) the arch height parameter, (A3) the arch length parameter, (C1) the sports item requirement and (C2) the medical disease name requirement; and a mid-foot bridge width parameter, which can be acquired by adaptation according to (C3) the footwear type.

For example, the transverse arch pad parameter can be accordingly divided into size types of low, present, no+3 mm and 6 mm.

For example, the inner arch parameter can be accordingly divided into size types of low, normal and high, so as to correspond to conditions adapted to such as flatfoot, plantar fasciitis, hallux valgus, high arches, genu varum (bow-shaped legs), and genu valgum (knocked knees).

For example, the outer arch parameter can be accordingly divided into size types of low, normal and high.

For example, the arch pad parameter can be accordingly divided into size types of XS, S, M, L, XL and XL+S.

For example, mid-foot bridge width parameter can be accordingly divided into size types of width, medium and narrow.

In one embodiment, the insole prescription includes an angle adaptation angle including at least one of a group consisting of a forefoot locally heightening parameter and a rearfoot locally heightening parameter, wherein the front locally heightening parameter can be acquired by adaptation according to (A2) the hallux valgus angle parameter, and the rearfoot locally heightening parameter can be acquired by adaptation according to (A1) the heel feature parameter.

For example, assuming that the hallux valgus angle is:
level 1 of over 10 degrees, the inner side is then heightened by 2 degrees;
level 2 of over 15 degrees, the inner side is then heightened by 3 degrees;
level 3 of over 20 degrees, the inner side is then heightened by 6 degrees; and
level 4 of over 25 degrees, the inner side is then heightened by 0 degrees.

For example, to increase the height on the inner side or the outer by 2 to 9 degrees side according to (A1) the heel feature parameter of a rearfoot, the two are added and divided by two as a support angle if a dynamically measured angle is greater than an angle of standing still. For greater than 3 degrees, a tilt angle of a heel line is accordingly used as angle to be adjusted for the insole prescription, and if the heel tilts inward, the inner side is heightened according to the tilt angle x80% that is rounded to the nearest whole number as a wedge angle. For over 10 degrees, an angle in the same direction as the forefoot is added by one level as a support at a forefoot angle.

In one embodiment, the insole prescription includes a pressure reduction adaptation angle, including at least one of a group consisting of the forefoot reinforcement parameter and the rearfoot reinforcement parameter, wherein the forefoot reinforcement parameter can be acquired by integration and adaptation according to (A2) the hallux valgus angle parameter and (C5) the user requirement feature, and the rearfoot reinforcement parameter can be acquired by integration and adaptation according to (A1) the heel feature parameter, (C1) the sports item requirement, (C2) the medical disease name requirement and (C5) the usage requirement feature.

The forefoot reinforcement parameter can be divided into pattern, material and size. The pattern can be further divided into square, trapezoid and circle. The material can be further divided into PORON and EVA30. The size can be further divided into large, medium and small.

The rear reinforcement parameter can be divided into pattern, material and size. The pattern can be further divided into a circle, a U-shape and a J-shape. The material can further be PORON. The size can be further divided into large, medium and small. For example, different sizes are suitable for different positions such as thumb knuckles, forefoot or rearfoot. The U-shape and J-shape are suitable for pressure reduction requirements for bone spurs, prolonged standing and plantar fasciitis, and the circle is suitable for pressure reduction requirements for prolonged standing, heel pain, plantar fasciitis and sports.

In one embodiment, the insole prescription includes a thickness adaptation parameter, which can be acquired by adaptation according to the usage requirement feature including (C1) the sports item requirement and (C2) the medical disease name requirement; for example, a sports thin elastic insole suitable for (C1) the sports item requirement, and a correction thick supportive insole suitable for (C2) the medical disease name requirement.

In one embodiment, the insole prescription includes a heel shape adaptation parameter, which can be acquired by adaptation according to the usage requirement feature including (C1) the sports item requirement and (C4) the age information. For example, the heel shape adaptation parameter can be a heel cup shape, and can be divided into types of cup shape present, low cup shape and no cup shape.

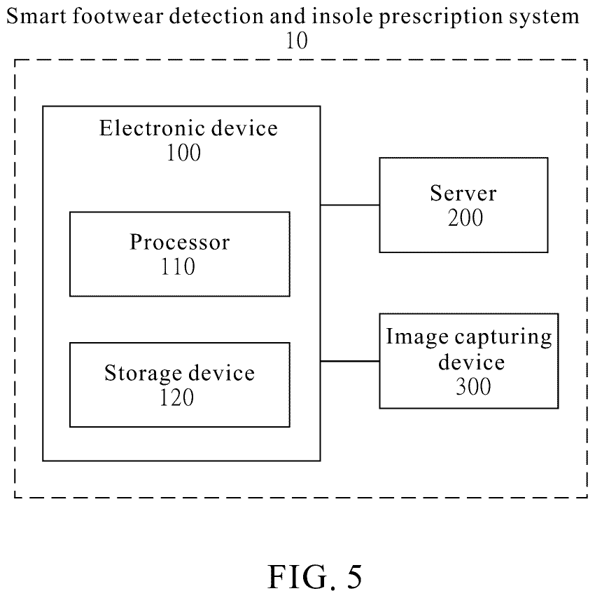

FIG. 5 shows a block diagram of a structure of a smart footwear detection and insole prescription system according to yet another embodiment of the present disclosure. A smart footwear detection and insole prescription system 10 is provided according to another embodiment of the present disclosure, the system operable to perform the smart footwear detection and insole prescription method above. The smart footwear detection and insole prescription system 10 includes a server 200 and an electronic device 100. The electronic device 100 is operable to be electrically connected to the server 200. The electronic device 100 includes, for example but not limited to, a processor 110 and a storage device 120. In one embodiment, the electronic device 100 further includes an input module and an output module (not shown) to provide a visual and/audio user interface, such as a display, a touch screen, a projector, a speaker, telephone audio, a keyboard, a mouse, a touch screen, motion detection and voice recognition. The storage device 120 has multiple program instructions stored therein. When the processor 110 executes the program instructions, steps S101 to S103, steps S201 to S206, steps S301 to S306 and steps S401 to S407 in FIG. 1 to FIG. 4 can be performed. The electronic device 100 can be a smartphone, a desktop computer, a laptop computer, a tablet computer, a workstation or a cloud server. The server 200 can be a cloud computing server connected via a network. The processor 110 can be such as a processing unit of a cloud server or a main processor of the electronic device 100. The storage device 120 can be a memory or a hard drive in the electronic device 100, for example, a flash memory or other types of memories. The storage device 120 stores program instructions executable by the processor 110. When the program instructions are executed by the processor 110 of the smart footwear detection and insole prescription system 10, the steps of the smart footwear detection and insole prescription method above can be performed. Moreover, a user can also directly input the parameters of the foot posture feature via the input module for subsequent analysis and providing of the insole prescription; that is, the input is not limited to images.

A non-transitory electronic device-readable storage medium adapted for a smart footwear detection and insole prescription is provided according to another embodiment of the present disclosure. The storage medium has multiple program instructions stored therein to enable an electronic device, after executing the program instructions, to perform the steps of the smart footwear detection and insole prescription method above.

FIG. 6 shows an illustrative schematic diagram of acquiring a foot posture feature according to an embodiment of the present disclosure, and FIG. 7 shows a footwear usage feature according to an embodiment of the present disclosure. FIG. 6 shows a determination method for the heel image information of the foot posture feature. A line at a midpoint of a connecting line between bone protrusions on both sides of an ankle is used as a tilt reference line, which is compared with a normal line perpendicular to the horizontal ground, and an included angle between the two is the tilt angle of the heel. FIG. 7 shows an item analyzed by the footwear usage feature, and includes a footwear heel image captured from a direction of the heel so as to determine wear conditions of a bottom of the footwear and a heel tilt angle thereof, and a footwear bottom image captured from the bottom of the footwear so as to determine wear conditions of the bottom of the footwear and a relationship between the levels of wear and regions of wear.

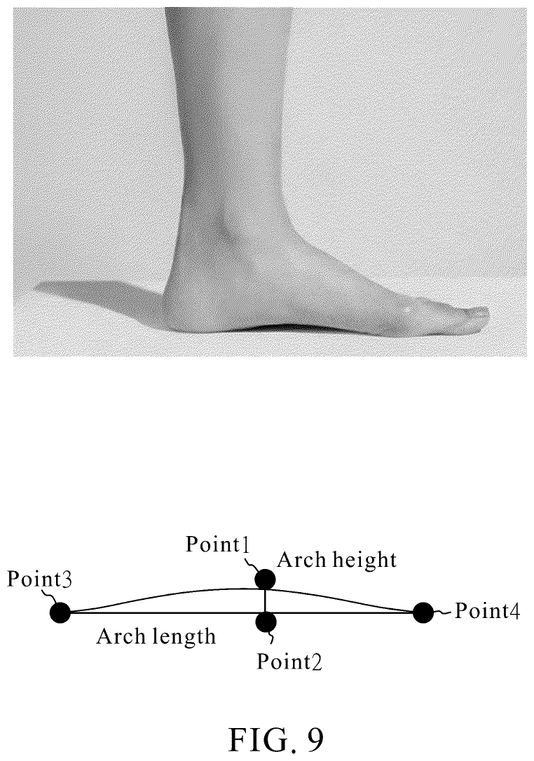

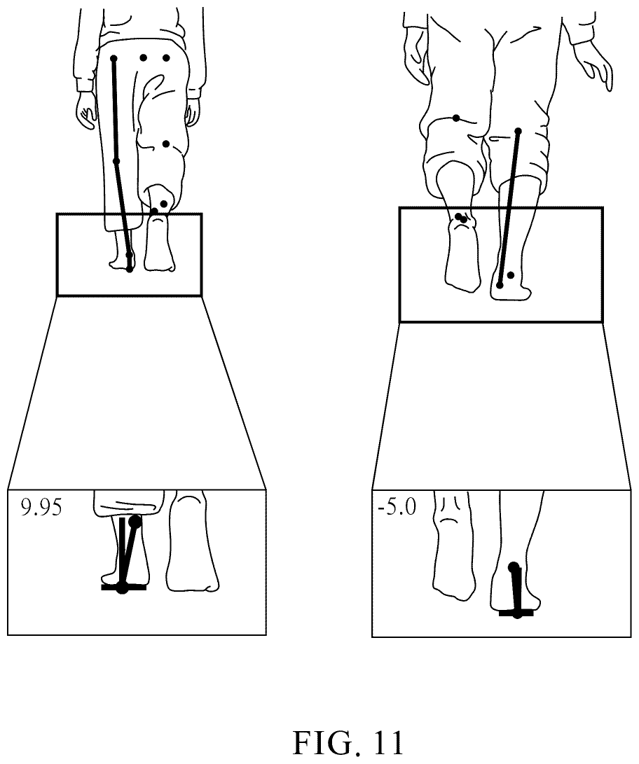

FIG. 8 shows an illustrative schematic diagram of acquiring a hallux valgus angle parameter according to an embodiment of the present disclosure. FIG. 9 shows an illustrative schematic diagram of acquiring an arch height parameter and an arch length parameter according to an embodiment of the present disclosure, wherein the lower half of FIG. 9 shows an enlarged partial schematic diagram of the arch feature in the upper half of FIG. 9. FIG. 10 shows an illustrative schematic diagram of acquiring a foot length parameter, a foot width parameter and a foot sole circumference parameter according to an embodiment of the present disclosure. FIG. 11 shows a schematic diagram of gait analysis of a heel dynamic image according to an embodiment of the present disclosure, and two frames of continuous gait are used for illustration, wherein the upper half of FIG. 11 shows posture analysis and feature points of lower limbs of continuous gait, the lower half of FIG. 11 shows heel feature parameters of a heel and heel image information, and the numerals are, for example, a tilt angle of a heel. It should be noted that, the feature points in the schematic diagram of gait analysis in the heel dynamic image are examples, and feature points for determining a heel tilt angle in practice includes, for example but not limited to, a midpoint between bone protrusions on both sides and a midpoint of a calcaneal line. Thus, the feature points shown in this schematic diagram are not necessarily at a midpoint position of a heel line in the schematic diagram.

Steps of a process of a smart footwear detection and insole prescription method according to an embodiment of the present disclosure are specifically described below.
1. A system starts to be operated.
2. Image capturing or video recording is selected, or uploading image information such as pictures or videos is selected.
3. Images are uploaded to a server to perform image recognition, wherein feature points in the images can be determined and recognized by the machine learning model above.
4. Coordinates of each point are acquired, and the system acquires a foot posture feature, and the coordinates of each point are returned, for example, a sole front endpoint, a sole rear endpoint and an arch endpoint.
5. According to the coordinates, positions of the individual points are displayed on an image for a user to view via a user interface or a display device.
6. According to the positions of the points, a line is drawn on the image for subsequent angle analysis.
7. Various angles are calculated to draw lines on the image.
8. A user learns his/her foot posture feature according to information in the image.
9. The system provides a corresponding insole prescription according to the foot posture feature.
10. The process of the system ends.

During a process of image capturing or video recording, any image capturing module desired can be used, for example but not limited to, a camera lens of a cellphone, a video recorder, a webcam or a laptop computer.

The acquiring of the heel image information can be image capturing or video recording, and can be divided into still image capturing or dynamic image capturing. The term “still” means that when the body is standing still, a heel posture of standing still can be obtained by capturing the heel in a forward direction from the back. On the other hand, the term “dynamic” means that when the body is in a state of walking or running, a heel posture of the heel landing on the ground during the dynamic state of walking or running is obtained from a video capture/recorded in a forward direction from the back. In a dynamic image when a non-landing foot is at a highest point, it is considered that a landing foot is in a state of receiving a maximum force. This frame is used for image and angle analysis. As shown in FIG. 11, two frames of continuous gait are shown as an example in FIG. 11. The upper half of FIG. 11 is a schematic diagram of posture analysis and feature points of overall lower limbs of continuous gait, the lines are connecting lines of the feature points, and the feature points are, for example but not limited to, a midpoint of hips, midpoints of hips on both sides, an inner side of the knee, the ankle and the heel of sole. The lower half of FIG. 11 is an enlarged partial diagram of the upper half, and shows heel feature parameters of the heel and the heel image information, wherein the numerals are, for example, a tilt angle of the heel, wherein the tilt angle of the heel is as described above. Thus, the dynamic image of a body during walking or running can be acquired, a key frame can be determined by determining the highest point as described above, and the heel feature parameters can be acquired by analysis. It should be noted that FIG. 11 is merely an example, and the feature points are not necessarily at middle positions of the heel as shown by the lines. In addition, the analysis for dynamic influences is not limited to key frames, and individual frames of the entire dynamic image can also be determined and analyzed according to requirements. In one embodiment, the analysis of the posture, feature points and heel feature points of continuous gait can be accomplished by an artificial intelligence model, so as to provide a large amount of foot image information and the corresponding foot posture features as training data, hence further achieving uniform, standardized, automatic, fast, stable and highly repetitive interpretation for the foot posture features, readily providing a stable and reliable insole prescription subsequently.

In one embodiment, the analysis of the foot image information also includes analysis of the hallux valgus angle parameter. Next, exemplary steps for acquiring the hallux valgus angle parameter are to be described below. Referring to FIG. 8, the process thereof is specifically as follows.
1. Both feet are put to step on a uniform background (for example, white paper), and a camera is hand held in a perpendicular manner to capture a picture from up to down.
2. After converting picture into a binary image, pixels are scanned from top to bottom, from right to left of the left foot, and from left to right of the right foot, until a point as a non-pixel value scanned is a stop point, that is, point 1.
3. After converting picture into a binary image, pixels are scanned from top to bottom, from right to left of the left foot, and from left to right of the right foot, until a point as a non-pixel point scanned is the stop point. The taken point is recorded, and 1 is added to the Y axis. Scanning from right to left of the left foot and from left to right of the right foot is continued, until the X axis of the 2 previous points found is smaller than the X axis of the current point but larger than the X axis of the previous 2 points is found. All points are compared, the largest point of the X axis is point 2, and the smallest point of the Y axis is point 3.
4. A straight line is drawn between point 1 and point 2, that is, line 1, and a straight line is drawn between point 2 and point 3, that is, line 2.
5. An included angle between line 1 and line 2 is calculated as the thumb angle.

The scanning refers to scanning one point after another in a two-dimensional image for image feature determination.

In one embodiment, the analysis of the foot image information also includes analysis of the arch height parameter and the arch length parameter. Next, exemplary steps for acquiring the arch height parameter and the arch length parameter are to be described below. Referring to FIG. 9, the process thereof is specifically as follows.
1. A foot is put to step on a uniform background (for example, white paper), and a camera is hand held at a same horizontal line as that the feet, and a picture of an inner side of the foot is captured. It should be noted that, the color of the background is kept as simple as possible during image capturing, for example, a uniform background, so as to prevent shadow interference.
2. The picture is converted into grayscale and binarized to generate a binarized picture.
   Scanning is performed from top to bottom, until a certain point of which Y is a maximum value is scanned, that is, point 1.
   Scanning is performed from bottom to top, until a certain point of which Y is a minimum value is scanned, that is, point 2.
   Scanning is performed from left to right, until a certain point of which X is a minimum value is scanned, that is, point 3.
Scanning is performed from right to left, until a certain point of which X is a maximum value, that is, point 4.
3. A difference in a distance between point 1 and point 2 is converted into centimeters, and this the arch height.
4. A difference in a distance between point 3 and point 4 is converted into centimeters, and this the arch length.

It should be noted that, when the scanning and image processing above are performed, attention should be paid to a conversion relationship between pixels of an image and a real length, so as to prevent a size error during the analysis and determination of the foot feature parameter.

In one embodiment, the analysis of the foot image information also includes analysis of the foot length parameter, the foot width parameter and/or the foot sole circumference parameter. Next, exemplary steps for acquiring the foot length parameter, the foot width parameter and/or the foot sole circumference parameter are to be described below. Referring to FIG. 10, the process thereof is specifically as follows.
1. A picture is captured by using a uniform background (for example, white paper), and scanning is performed from top to bottom and left to right to identify a point of which X is a minimum value, that is, point 1.
2. A picture is captured by using a uniform background (for example, white paper), and scanning is performed from top to bottom and right to left to identify a point of which X is a minimum value, that is, point 2.
3. A distance between point 1 and point 2 is the forefoot width.
4. A picture captured by using a grid pattern (for example, white paper printed with grids) is scanned from bottom to top and left to right to identify a point of which X is a minimum value, that is, point 3.
5. A picture captured by using a grid pattern (for example, white paper printed with grids) is scanned from bottom to top and right to left to identify a point of which X is a minimum value, that is, point 4.
6. A distance between point 3 and point 4 is the foot length.
7. A picture captured by using a grid pattern (for example, white paper printed with grids) is scanned upward from about ⅓ of the foot to a highest point of the foot, that is, point 5.
8. A distance between point 4 and point 5 is the forefoot height.

It should be noted that, the use of a grid pattern (for example, white paper printed with grids) and a uniform background (for example, white paper) used as a background for image capturing can be alternately used according to actual requirements so as to acquire clear images; however, the present disclosure is not limited to the example above.

With the smart footwear detection and insole prescription method and system of the present disclosure, by determining foot posture features of foot image information and accordingly providing an insole prescription, an insole prescription for a properly fitting and functional insole and/or providing rehabilitation and health care for foot diseases and a further customized insole can be provided. Moreover, the insole prescription with improved comfort, reduced burdens on muscles and enhanced sports performance is provided according to different factors, such as object foot features, objective footwear features, object sensations and usage requirements, thereby reducing burdens of sports or meeting requirements of injury prevention for general users.

Meanwhile, the smart footwear detection and insole prescription method and system of the present disclosure enable users to conveniently and easily carry out a full footwear detection and insole prescription procedure in a comfortable household area while overcoming venue restrictions and reducing human intervention, thereby improving convenience, accessibility, reliability and efficiency in providing the insole prescription. Accordingly, the present disclosure is capable of automatically, quickly, stably and accurately detecting and analyzing feet, footwear and subjective requirements of a user to accordingly provide properly adapted insole prescriptions, and is applicable in various venues such as personal household areas. According to foot image information captured by a user or other individuals using a common electronic device, the user is able to at all times and in all places analyze and acquire foot posture features, and an insole prescription adapted to a foot of the user can be automatically generated. With the insole prescription, a customized insole meeting user requirements of pressure reduction, buffering, force dispersion, support, fixation and stability can be subsequently manufactured.

The present invention is described by way of the preferred embodiments above. A person skilled in the art should understand that, these embodiments are merely for describing the present invention are not to be construed as limitations to the scope of the present invention. It should be noted that all equivalent changes, replacements and substitutions made to the embodiments are to be encompassed within the scope of the present invention. Therefore, the protection of the present disclosure should be accorded with the broadest interpretation of the appended claims, so as to encompass all modifications and similar arrangements and processes.

While the present disclosure has been described by means of specific embodiments, numerous modifications and variations could be made thereto by those skilled in the art without departing from the scope and spirit of the present disclosure set forth in the claims.

What is claimed is:

1. A smart footwear detection and insole prescription method, adapted to be performed on an electronic device, the smart footwear detection and insole prescription method comprising:
receiving foot image information;
acquiring a foot posture feature based on the foot image information; and
generating an insole prescription according to the foot posture feature.

2. The smart footwear detection and insole prescription method according to claim 1, wherein the foot image information comprises heel image information, wherein the heel image information comprises a heel dynamic image and a heel still image, wherein the foot posture feature comprises a heel feature parameter, and wherein the heel feature parameter is acquired based on the heel image information.

3. The smart footwear detection and insole prescription method according to claim 1, wherein the foot posture feature comprises an arch height parameter and an arch length parameter, wherein the arch height parameter and the arch length parameter are acquired by an image capturing module, and wherein a grid pattern is used as an image capturing background when the arch height parameter and the arch length parameter are acquired by the image capturing module.

4. The smart footwear detection and insole prescription method according to claim 1, wherein the foot posture feature comprises a hallux valgus angle parameter.

5. The smart footwear detection and insole prescription method according to claim 4, wherein the hallux valgus angle parameter is acquired by an image capturing module, and wherein a uniform background is used as an image capturing background when the hallux valgus angle parameter is acquired by the image capturing module.

6. The smart footwear detection and insole prescription method according to claim 1, wherein the foot posture feature comprises at least one of a group consisting of a foot length parameter, a foot width parameter, a foot sole circumference parameter and a body posture feature.

7. The smart footwear detection and insole prescription method according to claim 1, wherein the operation of acquiring a foot posture feature based on the foot image information is performed by an artificial intelligence model.

8. The smart footwear detection and insole prescription method according to claim 1, further comprising:
receiving footwear detection image information;
acquiring footwear usage feature based on the shoe image information; and
adjusting the insole prescription according to the shoe usage feature.

9. The smart footwear detection and insole prescription method according to claim 8, wherein the detection image information comprises a footwear heel image and a footwear bottom image.

10. The smart footwear detection and insole prescription method according to claim 1, further comprising:
receiving a usage requirement input;
acquiring a usage requirement feature based on the user requirement input; and
adjusting the insole prescription according to the usage requirement feature.

11. The smart footwear detection and insole prescription method according to claim 1, wherein the insole prescription is acquirable according to the foot posture feature, a footwear usage feature and a usage requirement feature.

12. The smart footwear detection and insole prescription method according to claim 1, wherein the insole prescription comprises a support adaptation parameter comprising an adaptive and adjustable insole arch height parameter, the insole arch height parameter comprising at least one of a group consisting of:
a transverse arch parameter, acquirable by integration and adaption according to a foot length parameter, a foot width parameter, a foot sole circumference parameter, a body posture feature and a sports requirement item;
an inner arch parameter, acquirable by integration and adaption according to an arch height parameter, an arch length parameter and a medical disease name requirement;
an outer arch parameter, acquirable by adaptation according to the sports item requirement;
an arch pad parameter, acquirable by integration and adaption according to the arch height parameter, the arch length parameter, the sports item requirement and the medical disease name; and
a mid-foot bridge width parameter, acquirable by adaptation according to a footwear type.

13. The smart footwear detection and insole prescription method according to claim 1, wherein the insole prescription comprises an angle adaptation parameter comprising at least one of a group consisting of a forefoot locally heightening parameter and a rearfoot locally heightening parameter, wherein the forefoot locally heightening parameter is acquirable by adaptation according to a hallux valgus angle parameter, and wherein the rearfoot locally heightening parameter is acquirable by adaptation according to a heel feature parameter.

14. The smart footwear detection and insole prescription method according to claim 1, wherein the insole prescription comprises a pressure reduction adaptation parameter comprising at least one of a group consisting of a forefoot reinforcement parameter and a rearfoot reinforcement parameter, wherein the forefoot reinforcement parameter is acquirable by integration and adaptation according to a hallux valgus angle parameter and a usage requirement feature, and wherein the rearfoot reinforcement parameter is acquirable by integration and adaptation according to a heel feature parameter, a sports item requirement, a medical disease name requirement and the usage requirement feature.

15. The smart footwear detection and insole prescription method according to claim 1, wherein the insole prescription comprises a thickness adaptation parameter, which is acquirable by adaptation according to a usage requirement feature comprising a sports item requirement and a medical disease name requirement.

16. The smart footwear detection and insole prescription method according to claim 1, wherein the insole prescription comprises a shape adaptation parameter, which is acquirable by adaptation according to a usage requirement feature including a sports item requirement and age information.

17. A smart footwear detection and insole prescription system, comprising:
a server; and
an electronic device, operable to be electrically connected to the server, the electronic device comprising a processor and a storage device, the storage device having multiple program instructions stored therein, the processor capable of performing the steps of the smart footwear detection and insole prescription method according to claim 1 after executing the program instructions.
